(19) United States
(12) Patent Application Publication
(10) Pub. No.: US 2010/0286220 A1
(43) Pub. Date: Nov. 11, 2010

(54) HNF4alpha ANTAGONISTS AND METHODS OF USE

(75) Inventors: Fred Levine, Del Mar, CA (US); Mark Mercola, Del Mar, CA (US); Suzette Farber-Katz, Rancho Santa Margarita, CA (US); Marcia I. Dawson, Los Altos, CA (US); Pamela Itkin-Ansari, Carlsbad, CA (US)

Correspondence Address: PATENT CORRESPONDENCE;ARNALL GOLDEN GREGORY LLP, 171 17TH STREET NW, SUITE 2100, ATLANTA, GA, 30363, US

(73) Assignees: Burnham Institute for Medical Research, La Jolla, CA (US); Regents of the University of California, Oakland, CA (US)

(21) Appl. No.: 12/771,813

(22) Filed: Apr. 30, 2010

Related U.S. Application Data
(60) Provisional application No. 61/174,450, filed on Apr. 30, 2009.

(51) Int. Cl. A61K 31/4184; A61P 3/00; A61P 3/06; A61P 31/20; A61P 35/00; C40B 30/04

(52) U.S. Cl. 514/395; 506/9

(57) ABSTRACT

Disclosed are methods and compositions relating to antagonists of HNF4α.

### CROSS-REFERENCE TO RELATED APPLICATIONS

This application claims benefit of U.S. Provisional Application No. 61/174,450, filed Apr. 30, 2009. Application No. 61/174,450, filed Apr. 30, 2009, is hereby incorporated herein by reference in its entirety.

### STATEMENT REGARDING FEDERALLY SPONSORED RESEARCH

This invention was made with government support under NIH grants R21 NS057001 and 3 R01 DK055283-08S1. The government has certain rights in the invention.

### REFERENCE TO SEQUENCE LISTING

The Sequence Listing submitted Apr. 30, 2010 as a text file named “24520_{—}14_{—}8403_{—}2010_{—}04_{—}30_AMD_AFD_Sequence_Listing.pdf,” created on Apr. 27, 2010, and having a size of 958 bytes is hereby incorporated by reference pursuant to 37 C.F.R. §1.52(e)(5).

### FIELD OF THE INVENTION

The disclosed invention is generally in the field of hepatocyte nuclear factor-4α (HNF4α) and specifically in the field of antagonists of HNF4α.

### BACKGROUND OF THE INVENTION

Hepatitis B virus (HBV) is the infectious agent that triggers hepatitis B. Chronic HBV affects about 350 million people worldwide. Once an individual is infected, HBV targets the liver eventually causing scarring of the liver (cirrhosis) and liver failure. According to the World Health Organization, HBV is 100 times more infectious than human immunodeficiency virus (HIV) and is readily transmitted through blood and bodily fluids. There is no known cure for HBV, and even with new treatments available, each year it is estimated that 5000 Americans and one million individuals worldwide die from hepatitis's major sequelae: cirrhosis and hepatocellular carcinoma. Furthermore, viral hepatitis is the single most important cause of liver disease. Many infectious agents, including hepatitis A, B, C, D, and E viruses, can cause viral hepatitis. HBV is unusual among DNA viruses because its replication involves reverse transcription of an RNA intermediate. Infection with HBV induces a broad spectrum of liver diseases, including acute hepatitis (that can lead to fulminate hepatic failure) as well as chronic hepatitis, cirrhosis, and heptocellular carcinoma (HCC). There is an effective preventive vaccine, however, 316,000 new cases of HEV-associated cancers are still diagnosed each year. WHO, World Health Report 1996: Fighting Disease, Fostering Development (World Health Organization, Geneva, 1996).

Acute HBV infection, while usually self-limited, can cause fulminant disease, as well as progressing to a chronic state associated with low-level but persistent viral replication. Similar to HIV, HBV depends on an error-prone reverse transcriptase for virus replication. Thus, the virus is susceptible to reverse transcriptase (RT) inhibitors, which are the mainstay of therapy along with alpha-interferon, a non-specific immune modulator. Similar to HIV, resistance to RT inhibitors is a major problem for HBV therapy. While newer generations of HBV polymerase inhibitors appear to be less prone to select for resistance, the fact that they share the same fundamental mechanism of action makes the eventual development of resistance almost inevitable. HIV therapy advanced with the development of drugs acting on additional targets, e.g., protease and integrase inhibitors. To achieve similar progress with HBV, there is a critical need for additional therapeutic targets. Mari Inada and Osamu Yokosuka. Current antiviral therapies for chronic hepatitis B. Hepatology Research 2008; 38: 535-542.

There are several known risk factors for atherosclerotic cardiovascular disease (ASCVD), the major cause of mortality in the Western world. One key risk factor is hyperlipidemia, which is the presence of elevated levels of lipids in blood plasma. Various epidemiological studies have demonstrated that drug-mediated lowering of total cholesterol (TC) and low-density lipoprotein (LDL) cholesterol (LDL-C) is associated with a significant reduction in cardiovascular events. The National Cholesterol Education Program's (NCEP's) updated guidelines recommend that the overall goal for high-risk patients is to achieve less than 100 mg/dL of LDL, with a therapeutic option to set the goal for such patients to achieve a LDL level less than 70 mg/dL.

One form of hyperlipidemia is known as hypertriglyceridemia and results in the presence of elevated amounts of triglycerides in the blood. Although triglycerides are necessary for good health, higher-than-normal triglyceride levels, often are associated with known risk factors for heart disease.

Another form of hyperlipidemia, known as hypercholesterolemia, which is the presence of elevated amounts of cholesterol in the blood, is a polygenic disorder. Modifications in lifestyle and conventional drug treatment are usually successful in reducing cholesterol levels. However, in some cases, as in familial hypercholesterolemia (FH), the cause is a monogenic defect. Treatment of a patient with FH can be more challenging because the levels of LDL-C remain elevated despite aggressive use of conventional therapy.

For example, one type of FH, homozygous familial hypercholesterolemia (hoFH), is a serious life-threatening genetic disease caused by homozygosity or compound heterozygosity for mutations in the low density lipoprotein (LDL) receptor. Patients with hoFH typically have total plasma cholesterol levels over 400 mg/dL resulting in premature atherosclerotic vascular disease. When left untreated, most patients develop atherosclerosis before age 20 and generally do not survive past age 30. Moreover, patients diagnosed with hoFH are largely unresponsive to conventional drug therapy and have limited treatment options. Specifically, treatment with statins, which reduce LDL-C by inhibiting cholesterol synthesis and upregulating the hepatic LDL receptor, have negligible effect in patients whose LDL receptors are non-existent or defective. A mean LDL-C reduction of only less than about 20% has been recently reported in patients with genotype-confirmed hoFH treated with the maximal dose of statins (atorvastatin or simvastatin administered at 80 mg/day). The addition of ezetimibe at 10 mg/day to this regimen resulted in a total reduction of LDL-C levels of 27%, which is still far from optimal. Non-pharmacological options have also been tested, including surgical interventions, such as portacaval shunt and ileal bypass, and orthotopic liver transplantation, but with clear disadvantages and risks. Therefore, there is a tremendous unmet medical need for new medical therapies for hoFH.

The microsomal triglyceride transfer protein (MTP) catalyzes the transport of triglyceride (TG), cholesteryl ester (CE), and phosphatidylcholine (PC) between small unilamellar vesicles (SUV). Wetterau & Zilversmit, Chem. Phys. Lipids 38, 205-22 (1985). When transfer rates are expressed as the percent of the donor lipid transferred per time, MTP expresses a distinct preference for neutral lipid transport (TG and CE), relative to phospholipid transport. The protein from bovine liver has been isolated and characterized. Wetterau & Zilversmit, Chem. Phys. Lipids 38, 205-22 (1985). It was demonstrated by Sharp et al., Nature (1993) 365:65 that the defect causing abetalipoproteinemia is in the MTP gene. This indicates that MTP is required for the synthesis of Apo B-containing lipoproteins such as VLDL, the precursor to LDL. It therefore follows that an MTP inhibitor would inhibit the synthesis of VLDL and LDL, thereby lowering levels of VLDL, LDL, cholesterol and triglyceride in humans.

Subjects with abetalipoproteinemia are afflicted with numerous maladies. Kane & Havel, supra. Subjects have fat malabsorption and TG accumulation in their enterocytes and hepatocytes. Due to the absence of TG-rich plasma lipoproteins, there is a defect in the transport of fat-soluble vitamins such as vitamin E. This results in acanthocytosis of erythrocytes, spinocerebellar ataxia with degeneration of the fasciculus cuneatus and gracilis, peripheral neuropathy, degenerative pigmentary retinopathy, and ceroid myopathy. Treatment of abetalipoproteinemic subjects includes dietary restriction of fat intake and dietary supplementation with vitamins A, E and K.

### BRIEF SUMMARY OF THE INVENTION

Disclosed are methods and compositions relating to antagonists of HNF4α. For example, disclosed herein is a method of for treating a subject exposed to hepatitis B virus, the method comprising administering to the subject a composition comprising an HNF4α antagonist.

Also disclosed is a method for treating a subject with undesired expression of one or more genes regulated via HNF4α, the method comprising administering to the subject a composition comprising an HNF4α antagonist.

Also disclosed is a method for treating or preventing a metabolic disorder in a subject, the method comprising administering to the subject a composition comprising an HNF4α antagonist.

Also disclosed is a method for identifying compounds that interact with HNF4α, the method comprising bringing into contact a test compound, an HNF4α antagonist, and HNF4α, and detecting unbound HNF4α antagonist, wherein a given amount of unbound HNF4α antagonist indicates a compound that interacts with HNF4α.

Also disclosed is a method for identifying compounds that affect HNF4α regulation, the method comprising bringing into contact BIM5078 and an HNF4α-regulated gene, and detecting changes in the expression of the HNF4α-regulated gene in the presence and absence of a test compound, wherein a difference in expression of the HNF4α-regulated gene in the presence of the test compound relative to expression of the HNF4α-regulated gene in the absence of the test compound indicates a compound that affects HNF4αregulation.

In some forms of the method the subject can exhibit hyperinsulinemia. In some forms of the method the subject can be a neonate. In some forms of the method the subject can have cancer, wherein the cancer expresses HNF4α. In some forms of the method the cancer can be hepatocellular carcinoma. In some forms of the method the cancer can be gastric cancer.

In some forms of the method the composition can be a BIM5078 composition. In some forms of the method the BIM5078 composition can further comprise a detectable agent linked to the BIM5078 compound. In some forms of the method the method can further comprise bringing into contact BIM5078 and an HNF4α-regulated gene, and detecting changes in the expression of the HNF4α-regulated gene in the presence and absence of the compound that interacts with HNF4α, wherein a difference in expression of the HNF4α-regulated gene in the presence of the compound that interacts with HNF4αrelative to expression of the HNF4α-regulated gene in the absence of the compound that interacts with HNF4α indicates a compound that affects HNF4α regulation.

In some forms of the method a decrease in the expression of the HNF4α-regulated gene in the presence of the compound that interacts with HNF4α relative to expression of the HNF4α-regulated gene in the absence of the compound that interacts with HNF4α indicates that the compound that interacts with HNF4α inhibits HNF4α. In some forms of the method an increase in the expression of the HNF4α-regulated gene in the presence of the compound that interacts with HNF4α relative to expression of the HNF4α-regulated gene in the absence of the compound that interacts with HNF4α indicates that the compound that interacts with HNF4α decreases inhibition of HNF4α by BIM5078. In some forms of the method the method can further comprise detecting changes in the expression of the HNF4α-regulated gene in the absence of BIM5078 and in the presence and absence of the compound that interacts with HNF4α, wherein an increase in expression of the HNF4α-regulated gene indicates that the compound that interacts with HNF4α increases expression of the HNF4α-regulated gene.

In some forms of the method a decrease in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation inhibits HNF4α. In some forms of the method an increase in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation decreases inhibition of HNF4α by BIM5078. In some forms of the method the method can further comprise detecting changes in the expression of the HNF4α-regulated gene in the absence of BIM5078 and in the presence and absence of the compound that affects HNF4α regulation, wherein an increase in expression of the HNF4α-regulated gene indicates that the compound that affects HNF4α regulation increases expression of the HNF4α-regulated gene. In some forms of the method the HNF4α-regulated gene can express a reporter protein.

In some forms of the method the metabolic disorder can be a lipid metabolic disorder. In some forms of the method the subject can be hyperlipidemic. In some forms of the method the metabolic disorder can be or can result in hyperlipidemia.

The HNF4α antagonist can be a BIM5078 compound.

The HNF4α antagonist or BIM5078 compound can be a compound having the structure of

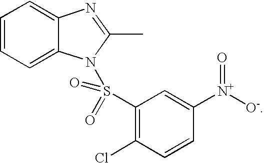

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a graph of insulin expression in the presence of increasing concentrations of 4-hydroxytamoxifen (40HT).

FIG. 2 shows screens targeting the Insulin promoter establishing hits including graphs of positive and negative controls from two data sets and the formula for calculating z′ (z prime) which is a measure of the ability to distinguish positives from negatives.

FIG. 3 shows the results of a primary confirmatory assay graphing the change in GFP expression versus BIM5078 concentration.

FIG. 4 shows the results of a secondary confirmatory assay graphing the change in endogenous insulin expression versus BIM5078 concentration.

FIGS. 5A, 5B and 5C show the structure of HNF4α. FIG. 5A shows the functional domains of HNF4α. FIG. 5B shows the three-dimensional structure of a binary rat HNF4α homodimer conjugated with a fatty acid with one subunit open and one subunit closed. FIG. 5C shows ternary human HNF4α monomer conjugated with fatty acid and SRC-1 peptide (the subunit is closed).

FIG. 6 shows the HNF4α ligand-binding pocket contact of fatty acid and the docked pose of BIM5078 superposed. This clearly shows the similarity of contacts for the two ligands and highlights unique contacts of BIM5078. BIM5078 carbons are in medium greytone; C₁₄-fatty acid carbons and LBD interacting-residue carbons, light graytone. Other, ligand and residue atoms colored as follows: nitrogen, dark graytone; oxygen, medium greytone; chlorine; light graytone.

FIG. 7 shows a surface representation of the HNF4α ligand-binding pocket as found in the crystal structure of the human HNF4α ligand-binding domain complexed with a C₁₄-fatty acid (PDB Code: 1pzl) (left panel) and the docked pose of BIM5078 superposed (right panel).

FIG. 8 shows graphs of the change in endogenous insulin expression versus BIM5078 concentration in the presence of low 4-hydroxytamoxifen (40HT) (left panel) and high 4-hydroxytamoxifen (right panel).

FIG. 9 shows the effect of BIM5078 on HBV surface antigen levels. BIM5078 decreased the HBsAg protein expression in Huh7 cells transfected with wild type HBV construct (pWTD). The HBsAg is seen as doublet bands with different glycosylation forms (GP33 and GP36).

FIGS. 10A and 10B show BIM5078 binding to HNF4α ligand-binding domain (LBD). A. Binding of BIM5078 was analyzed by quenching of intrinsic fluorescence of rat HNF4α LBD aromatic amino acids Tyr/Trp. The IQ for binding of BIM5078 to HNF4α was 11.9 nM. B. BIM5078 was docked to rat HNF4α LBD (PDB Code: 1m7w) using the program BioMedCache. Superposition of docked pose of BIM5078 with the original fatty acid ligand shows that the oxygen of the nitro group of BIM5078 can form a strong salt bridge (ionic) interaction and hydrogen bond with HNF4α residues Arg 226 and Gly 237, respectively. Moreover, the chloro group of BIM5078 forms a hydrophobic interaction with the side chain of the binding-pocket residue Val 178.

### DETAILED DESCRIPTION OF THE INVENTION

The disclosed method and compositions can be understood more readily by reference to the following detailed description of particular embodiments and the Example included therein and to the Figures and their previous and following description.

HNF4α is a member of the nuclear hormone receptor super-family of transcription factors. In general, these factors require binding of specific ligands for transcriptional activation. HNF4α binds to DNA only as a homodimer. There is substantial controversy whether it is regulated by interactions with a ligand. When expressed either in mammalian or bacterial cells, HNF4α is invariably bound to a fatty acid. Transfection studies with HNF4α have found it to be transcriptionally active in the absence of added ligand. This has led to a model that is widely believed but poorly supported by data in which the ligand-binding pocket of HNF4α is constitutively occupied by a mixture of fatty acids that play a structural rather than regulatory role in maintaining the protein in a transcriptionally active state.

HNF4α binding sites are found in upstream regions of numerous genes (Table 1). The disclosed HNF4α antagonists can be used to affect the undesired expression of any gene regulated by HNF4α. As used herein, undesired expression is any expression that is undesired. For example, expression of a gene that causes and/or contributes to a disease or condition can be considered undesired expression. A variety of diseases and conditions are caused by and/or associated with disregulation of one or more genes regulated by HNF4α. For example, excess expression of human transferrin (HTF) is associated with hyperlipidemia. As another example, some forms of cancer, such as hepatocellular carcinoma and gastric cancer express HNF4α which causes abnormal expression of HNF4α-regulated genes that contribute to the cancerous state.

An HNF4α-regulated gene is a gene whose expression is directly altered or affected by HNF4α. This generally will be via interaction or a reduction of interaction of HNF4α with regulatory sequences in the gene. A gene that is indirectly regulated by HNF4α is not considered an HNF4α-regulated gene as used herein. Indirect regulation would be, for example, regulation of a gene by the gene product of another gene that is regulated by HNF4α.

TABLE 1
HNF4α Target Genes

| | |
|---|---|
| Gene Name | Full Gene Name |

Transport
Lipid and Retinol Transport

| | |
|---|---|
| ApoAI | apolipoprotein AI |
| ApoAII | apolipoprotein AII |
| ApoAIV | apolipoprotein AIV |
| ApoB | apolipoprotein B |
| ApoCII | apolipoprotein CII |
| ApoCIII | apolipoprotein CIII |
| ApoE | apolipoprotein E |
| CRBPII | cellular retinol-binding protein II |
| Fabpi | intestinal fatty acid binding protein |
| MTP | microsomal triglyceride transfer |

Other Serum Proteins

| | |
|---|---|
| Transferrin | transferrin |
| TTR | transthyretin |
| α-1-AT | alpha-1-anti-trypsin |
| SHBG | sex hormone-binding globulin |

Blood Maintenance

| | |
|---|---|
| Factor VII | Factor VII |
| Factor VII | Factor VII |
| Factor VIII | Factor VIII |
| Factor IX | Factor IX |
| Factor IX | Factor IX |
| Factor IX | Factor IX |
| Factor X | Factor X |
| EPO | erythropoietin |
| EPO | erythropoietin |
| ATIII | antithrombin III |
| ATIII | antithrombin III |
| ANG | angiotensinogen |
| ANG | angiotensinogen |

Liver Differentiation

| | |
|---|---|
| HNF-1α | hepatocyte nuclear factor 1 alpha |
| HNF6 | hepatocyte nuclear factor 6 |

Nutrient Metabolism
Lipid and Steroid Metabolism

| | |
|---|---|
| MCAD | medium-chain acyl CoA immune function |
| ACO | acyl-CoA oxidase |
| mitHMG-CoA | mitochondrial 3-hydroxy-3-methylglutaryl-CoA |
| HD | acyl-CoA hydratase-dehydrogenase |
| TB | 3-keto acyl-CoA thiolase B |
| ALDH3 | aldehyde dehydrogenase 3 |

Xenobiotic Metabolism

| | |
|---|---|
| CYP2A4 | cytochrome 2A4 |
| CYP2C1 | cytochrome 2C1 |
| CYP2C2 | cytochrome 2C2 |
| CYP2C3 | cytochrome 2C3 |
| CYP2C9 | cytochrome 2C9 |
| CYP2D6 | cytochrome 2D6 |
| CYP3A1 | cytochrome 3A1 |
| CYP3A23 | cytochrome 3A23 |
| CYP7A1 | cytochrome 7A1 |
| CYP7 | cytochrome 7 |
| DD/AKR | dihydrodiol dehydrogenase 4 |

Glucose metabolism

| | |
|---|---|
| PEPCK | phospho-enol-pyruvate carboxykinase |
| L-PK | liver-type pyruvate kinase |
| AldolaseB | aldolase B |

Amino Acid Metabolism

| | |
|---|---|
| TAT | tyrosine amino transferase |
| OTC | ornithine transcarbamylase |
| mitALDH2 | mitochondrial aldehyde dehydrogenase 2 |

Immune Function
Immune System

| | |
|---|---|
| Bf | factor B |
| MSP | macrophage stimulating protein |
| AMBP | alpha-1-microglobulin & bikunin |
| HBV enh I | hepatitis B virus enhancer I |
| HBV enh II | hepatitis B virus enhancer II |
| HBVnucleocapsid | hepatitis B virus nucleocapsid |
| WHVEnII | woodchuck hepatitis virus enhancer II |
| HIV LTR | human immunodeficiency virus long terminal repeat |

Growth Factors

| | |
|---|---|
| HGFL | hepatocyte growth factor-like protein |
| PRLR | prolactin receptor |
| GHR1a | growth hormone receptor 1a |

Cell Structure and Function

| | |
|---|---|
| BGP | biliary glycoprotein/CEA family |
| GCC | guanylyl cyclase C |

Direct Repeat 1 (DR1g)

(SEQ ID NO: 1)

5′-AGGTCA g AGGTCA-3′
Direct Repeat 2 (DR2aa)

(SEQ ID NO: 2)

| | |
|---|---|
| 5′-AGGTCA aa AGGTCA-3′ | |
| Final Consensus | G G G T C A A A G G T C A |
| (SEQ ID NO: 3) | A t C t g g T C t g |
| | a G g c |
| | t |

Hepatitis B virus (HBV) is the infectious agent that triggers hepatitis B. Chronic HBV affects about 350 million people worldwide. Once an individual is infected, HBV targets the liver eventually causing scarring of the liver (cirrhosis) and liver failure. There is no known cure for HBV, and even with new treatments available, each year it is estimated that 5000 Americans and one million individuals worldwide die from hepatitis's major sequelae: cirrhosis and hepatocellular carcinoma. Furthermore, viral hepatitis is the single most important cause of liver disease. HNF4α binds to multiple sites within the HBV genome, including the enhancer-I/X gene promoter and the nucleocapsid promoter. While a number of transcription factors are involved in HBV transcription including Fox A2, PPARα, HNF1α, and a number of ubiquitous factors, the only one that has been shown thus far to be absolutely required for HBV transcription is HNF4α. This has been demonstrated using siRNA to HNF4α and indicates that HNF4α inhibition will prevent virus production.

The microsomal triglyceride transfer protein (MTP) catalyzes the transport of triglyceride (TG), cholesteryl ester (CE), and phosphatidylcholine (PC) between small unilamellar vesicles (SUV). Wetterau & Zilversmit, Chem. Phys. Lipids 38, 205-22 (1985). When transfer rates are expressed as the percent of the donor lipid transferred per time, MTP expresses a distinct preference for neutral lipid transport (TG and CE), relative to phospholipid transport. The protein from bovine liver has been isolated and characterized. Wetterau & Zilversmit, Chem. Phys. Lipids 38, 205-22 (1985). It was demonstrated by D. Sharp et al., Nature (1993) 365:65 that the defect causing abetalipoproteinemia is in the MTP gene. This indicates that MTP is required for the synthesis of Apo B-containing lipoproteins such as VLDL, the precursor to LDL. It therefore follows that an inhibitor of MTP expression would inhibit the synthesis of VLDL and LDL, thereby lowering levels of VLDL, LDL, cholesterol and triglyceride in humans.

MTP is regulated by HNF4α (Sheena et al., J. Lipid Research 46:328-341 (2005). HNF4α is required for transcription of the MTP gene and so antagonism of HNF4α can reduce expression of MTP. Because of MTP's central role in the production of lipoproteins, the disclosed HNF4α can be used to reduce excess lipoprotein production and levels by reducing expression of MTP.

A high-throughput screen discovered that BIM5078 (1-[(2′-chloro-5-nitrophenyl)sulfonyl]-2-methyl-1H-benzimidazole) was a suitable treating agent for several diseases associated with an undesired expression of genes associated with the HNF4α receptor. BIM5078 was discovered to be a potent inhibitor of insulin gene transcription, in the high-throughput screen for small-molecule modulators of the same promoter. BIM5078 showed that it was a potent inhibitor of the orphan nuclear receptor HNF4α (Examples 1 and 2). HBV is highly dependent on HNF4α for the expression of viral gene products. BIM5078 is able to affect transcriptional activity of HNF4α-regulated consistent with a model in which the ligand-binding pocket of HNF4α plays a role in regulating the active state of the HNF4α. BIM5078 inhibits HBV transcription in vitro. The potent inhibition of HNF4α in vitro is demonstrated by showing its inhibition of HBV (Example 3).

Disclosed herein are HNF4α antagonists and BIM5078 compounds having the structure of

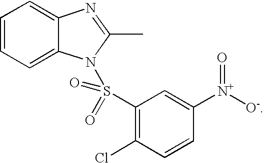

Disclosed are methods and compositions relating to antagonists of HNF4α. For example, disclosed herein is a method of treating a subject exposed to hepatitis B virus, the method comprising administering to the subject a composition comprising an HNF4αantagonist.

Also disclosed is a method for treating a subject with undesired expression of one or more genes regulated via HNF4α, the method comprising administering to the subject a composition comprising an HNF4α antagonist.

Also disclosed is a method for treating or preventing a metabolic disorder in a subject, the method comprising administering to the subject a composition comprising an HNF4α antagonist.

Also disclosed is a method for identifying compounds that interact with HNF4α, the method comprising bringing into contact a test compound, an HNF4α antagonist, and HNF4α, and detecting unbound HNF4α antagonist, wherein a given amount of unbound HNF4α antagonist indicates a compound that interacts with HNF4α.

Also disclosed is a method for identifying compounds that affect HNF4α regulation, the method comprising bringing into contact BIM5078 and an HNF4α-regulated gene, and detecting changes in the expression of the HNF4α-regulated gene in the presence and absence of a test compound, wherein a difference in expression of the HNF4α-regulated gene in the presence of the test compound relative to expression of the HNF4α-regulated gene in the absence of the test compound indicates a compound that affects HNF4α regulation.

In some forms of the method the subject can exhibit hyperinsulinemia. In some forms of the method the subject can be a neonate. In some forms of the method the subject can have cancer, wherein the cancer expresses HNF4α. In some forms of the method the cancer can be hepatocellular carcinoma. In some forms of the method the cancer can be gastric cancer.

In some forms of the method the composition can be a BIM5078 composition. In some forms of the method the BIM5078 composition can further comprise a detectable agent linked to the BIM5078 compound. In some forms of the method the method can further comprise bringing into contact BIM5078 and an HNF4α-regulated gene, and detecting changes in the expression of the HNF4α-regulated gene in the presence and absence of the compound that interacts with HNF4α, wherein a difference in expression of the HNF4α-regulated gene in the presence of the compound that interacts with HNF4α relative to expression of the HNF4α-regulated gene in the absence of the compound that interacts with HNF4α indicates a compound that affects HNF4α regulation.

In some forms of the method a decrease in the expression of the HNF4α-regulated gene in the presence of the compound that interacts with HNF4α relative to expression of the HNF4α-regulated gene in the absence of the compound that interacts with HNF4α indicates that the compound that interacts with HNF4α inhibits HNF4α. In some forms of the method an increase in the expression of the HNF4α-regulated gene in the presence of the compound that interacts with HNF4α relative to expression of the HNF4α-regulated gene in the absence of the compound that interacts with HNF4α indicates that the compound that interacts with HNF4α decreases inhibition of HNF4α by BIM5078. In some forms of the method the method can further comprise detecting changes in the expression of the HNF4α-regulated gene in the absence of BIM5078 and in the presence and absence of the compound that interacts with HNF4α, wherein an increase in expression of the HNF4α-regulated gene indicates that the compound that interacts with HNF4α increases expression of the HNF4α-regulated gene.

In some forms of the method a decrease in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation inhibits HNF4α. In some forms of the method an increase in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation decreases inhibition of HNF4α by BIM5078. In some forms of the method the method can further comprise detecting changes in the expression of the HNF4α-regulated gene in the absence of BIM5078 and in the presence and absence of the compound that affects HNF4α regulation, wherein an increase in expression of the HNF4α-regulated gene indicates that the compound that affects HNF4α regulation increases expression of the HNF4α-regulated gene. In some forms of the method the HNF4α-regulated gene can express a reporter protein.

In some forms of the method the metabolic disorder can be a lipid metabolic disorder. In some forms of the method the subject can be hyperlipidemic. In some forms of the method the metabolic disorder can be or can result in hyperlipidemia.

The HNF4α antagonist can be a BIM5078 compound.

The HNF4α antagonist or BIM5078 compound can be a compound having the structure of

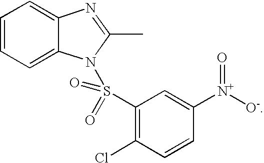

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an HNF4α antagonist is disclosed and discussed and a number of modifications that can be made to a number of molecules including the HNF4α antagonist are discussed, each and every combination and permutation of HNF4α antagonist and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

By “pharmaceutically acceptable” is meant a material that is not biologically, clinically or otherwise undesirable, i.e., the material can be administered to an individual along with the relevant active compound without causing clinically unacceptable biological effects or interacting in a deleterious manner with any of the other components of the pharmaceutical composition in which it is contained. By “pharmaceutically acceptable salt or acid form” is meant a form of a salt or acid compound that is not biologically, clinically or otherwise undesirable, i.e., the salt or acid form of the compound can be administered to an individual without salt or acid causing clinically unacceptable biological effects or interacting in a deleterious manner with any of the other components of the pharmaceutical composition in which it is contained.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The term “organic radical” defines a carbon containing moiety that forms a portion of a larger molecule, i.e. a moiety comprising at least one carbon atom, and can also often contain hydrogen atoms. Examples of organic radicals that comprises no heteroatoms are alkyls such as methyl, ethyl, n-propyl, or iso-propyl moieties, or cyclic organic radicals such as phenyl or tolyl moieties, or 5, 6, 7, 8-tetrahydro-2-naphthyl moieties. Organic radicals can and often do, however, optionally contain various heteroatoms such as halogens, oxygen, nitrogen, sulfur, phosphorus, or the like. Examples of organic residues include alkoxy or substituted alkoxy moieties such as methoxyl moieties or hydroxymethyl moieties, or in other examples trifluoromethyl moieties, mono or di-methyl amino moieties, carboxy moieties, formyl moieties, amide moieties, etc. An organic radical can have, for example, 1-26 carbon atoms, 1-18 carbon atoms, 1-12 carbon atoms, 1-8 carbon atoms, or 1-4 carbon atoms. Organic radicals often have a hydrogen bound to at least some of the carbon atoms of the organic radical. In some embodiments, an organic radical can contain 1-10, or 1-5 heteroatoms bound thereto.

The term “alkyl” denotes a hydrocarbon group or residue which is structurally similar to an alkane compound modified by the removal of one hydrogen from the non-cyclic alkane and the substitution therefore of a non-hydrogen moiety. “Normal” or “Branched” alkyls comprise a non-cyclic, saturated, straight or branched chain hydrocarbon moiety having from 1 to 12 carbons, or 1 to 8 carbons, 1 to 6, or 1 to 4 carbon atoms. Examples of such alkyl radicals include methyl, ethyl, n-propyl, iso-propyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms, i.e., C₁-C₄ alkyl.

Moreover, the term “alkyl” as used throughout the specification and claims is intended to include both “unsubstituted alkyls” and “substituted alkyls”, the later denotes an alkyl radical analogous to the above definition that is further substituted with one, two, or more additional organic or inorganic substituent groups. Suitable substituent groups include but are not limited to hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. It will be understood by those skilled in the art that an “alkoxy” can be a substituent of a carbonyl substituted “alkyl” forming an ester. When more than one substituent group is present then they can be the same or different. The organic substituent moieties can comprise from 1 to 12 carbon atoms, or from 1 to 6 carbon atoms, or from 1 to 4 carbon atoms. It will be understood by those skilled in the art that the moieties substituted on the “alkyl” chain can themselves be substituted, as described above, if appropriate.

The term “alkenyl” denotes an alkyl residue as defined above that also comprises at least one carbon-carbon double bond in the backbone of the hydrocarbon chain. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

The term “alkynyl” denotes a residue as defined above that comprises at least one carbon-carbon triple bond in the backbone of the hydrocarbon chain. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “cycloalkyl” denotes a hydrocarbon group or residue which is structurally similar to a cyclic alkane compound modified by the removal of one hydrogen from the cyclic alkane and substitution therefore of a non-hydrogen moiety. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

The term “substituted cycloalkyl” denotes a cycloalkyl residue as defined above that is further substituted with one, two, or more additional organic or inorganic groups that can include but are not limited to halogen, alkyl, substituted alkyl, hydroxyl, alkoxy, substituted alkoxy, carboxy, carboalkoxy, alkylcarboxamido, substituted alkylcarboxamido, dialkylcarboxamido, substituted dialkylcarboxamido, amino, mono-substituted amino or di-substituted amino. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

The term “cycloalkenyl” denotes a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like. The term “substituted cycloalkenyl” denotes a cycloalkyl as defined above further substituted with one or more groups selected from halogen, alkyl, hydroxyl, alkoxy, substituted alkoxy, haloalkoxy, carboxy, carboalkoxy, alkylcarboxamido, substituted alkylcarboxamido, dialkylcarboxamido, substituted dialkylcarboxamido, amino, mono-substituted amino or di-substituted amino. When the cycloalkenyl is substituted with more than one group, they can be the same or different. The organic substituent groups can comprise from 1 to 12 carbon atoms, or from 1 to 6 carbon atoms, or from 1 to 4 carbon atoms.

The term “alkoxy” as used herein denotes an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like

The term “mono-substituted amino” denotes a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH₃); ethylamino (—NH—CH₂CH₃), hydroxyethylamino (—NH—CH₂CH₂OH), and the like.

The term “di-substituted amino” denotes a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

The term “haloalkyl” denotes an alkyl residue as defined above, substituted with one or more halogens, preferably fluorine, such as a trifluoromethyl, pentafluoroethyl and the like.

The term “haloalkoxy” denotes a haloalkyl residue as defined above that is directly attached to an oxygen to form trifluoromethoxy, pentafluoroethoxy and the like.

The term “acyl” denotes a R—C(O)— residue having an R group containing 1 to 8 carbons. Examples include but are not limited to formyl, acetyl, propionyl, butanoyl, iso-butanoyl, pentanoyl, hexanoyl, heptanoyl, benzoyl and the like, and natural or un-natural amino acids.

The term “acyloxy” denotes an acyl radical as defined above directly attached to an oxygen to form an R—C(O)O— residue. Examples include but are not limited to acetyloxy, propionyloxy, butanoyloxy, iso-butanoyloxy, benzoyloxy and the like.

The term “aryl” denotes a ring radical containing 6 to 18 carbons, or preferably 6 to 12 carbons, comprising at least one aromatic residue therein. Examples of such aryl radicals include phenyl, naphthyl, and ischroman radicals. Moreover, the term “aryl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “aryl” can themselves be substituted, as described above, if appropriate.

The term “heteroaryl” denotes an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

The term “heterocyclyl” or “heterocyclic group” denotes a non-aromatic mono- or multi ring radical structure having 3 to 16 members, preferably 4 to 10 members, in which at least one ring structure include 1 to 4 heteroatoms (e.g. O, N, S, P, and the like). Heterocyclyl groups include, for example, pyrrolidine, oxolane, thiolane, imidazole, oxazole, piperidine, piperizine, morpholine, lactones, lactams, such as azetidiones, and pyrrolidiones, sultams, sultones, and the like. Moreover, the term “heterocyclyl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “heterocyclyl” can themselves be substituted, as described above, if appropriate.

The term “halo” or “halogen” refers to a fluoro, chloro, bromo or iodo group.

For the purposes of the present disclosure the terms “compound,” “analog,” and “composition of matter” stand equally well for the chemical entities described herein, including all enantiomeric forms, diastereomeric forms, salts, and the like, and the terms “compound,” “analog,” and “composition of matter” are used interchangeably throughout the present specification.

A “moiety” is part of a molecule (or compound, or analog, etc.). A “functional group” is a specific group of atoms in a molecule. A moiety can be a functional group or can include one or functional groups.

### A. HNF4α Antagonists

Disclosed are compounds and compositions comprising HNF4α antagonists. It has been discovered that a compound, referred to herein as the BIM5078 compound, is a useful HNF4α antagonist. The HNF4α antagonist or BIM5078 compound can be a compound having the structure of

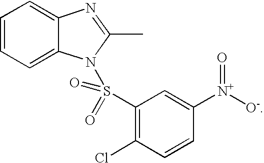

HNF4α antagonists can bind HNF4α in the fatty acid binding pocket of HNF4α. With this in mind, some HNF4α antagonists can be defined as compounds that competitively bind HNF4α in the presence of particular known antagonists HNF4α antagonists, such as BIM5078. The ability of a compound to antagonize HNF4α can be determined in any suitable manner. Methods for identifying and assessing the ability of compounds to bind to and/or antagonize HNF4α are described elsewhere herein.

HNF4α antagonists and BIM5078 compounds can be, for example, combined with other compounds and compositions, formulated into compositions, and conjugated with and/or coupled to other compounds. The use to which the HNF4α antagonists and BIM5078 compound is to be put can be used to determine suitable forms of HNF4α antagonist compositions and BIM5078 compositions. Those of skill in the art can make this determination based on the guidance provided herein and their own knowledge. The disclosed HNF4α antagonist can be used alone or in combination with one or more additional compounds or compositions. The disclosed compounds and compositions can comprise one or more HNF4α antagonists. In some forms, the disclosed HNF4α antagonist can be linked or coupled to one or more other compounds.

### B. Detectable Agent

A detectable agent is any compound, moiety, label or combination that can be detected. Labels are an example of a detectable agent. By “label” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is done can depend on the label. Exemplary labels include, but are not limited to, fluorescent labels, label enzymes and radioisotopes.

By “fluorescent label” is meant any molecule that an be detected via its inherent fluorescent properties. Suitable fluorescent labels include, but are not limited to 1,5-IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy-Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protei—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD—Lipophilic Tracer; DiD (DilC18(5)); DIDS; Dihydrorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyde Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP(S65T); GFP red shifted (rsGFP); GFP wild type non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

By “label enzyme” is meant an enzyme which can be reacted in the presence of a label enzyme substrate which produces a detectable product. Suitable label enzymes for use in the present methods include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products can be opaque, such as the reaction of horseradish peroxidase with tetramethyl benzedine, and can have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

In some instances, multiple fluorescent labels are used. In some aspects, at least two fluorescent labels are used which are members of a Fluorescence (Förster) Resonance Energy Transfer (FRET) pair. FRET refers to an energy transfer mechanism between two chromophores. A donor chromophore in its excited state can transfer energy by a nonradiative, long-range dipole-dipole coupling mechanism to an acceptor chromophore in close proximity (typically <10 nm).

An example of a FRET pair for biological use is a cyan fluorescent protein (CFP)-yellow fluorescent protein (YFP) pair. Both are color variants of green fluorescent protein (GFP). While labeling with organic fluorescent dyes requires troublesome processes of purification, chemical modification, and intracellular injection of a host protein, GFP variants can be easily attached to a host protein by genetic engineering.

Other FRET pairs (donor/acceptor) useful in the present methods include, but are not limited to, EDANS/fluorescein, IAEDANS/fluorescein, fluorescein/tetramethylrhodamine, fluorescein/LC Red 640, fluorescein/Cy 5, fluorescein/Cy 5.5 and fluorescein/LC Red 705.

In addition, labels can be indirectly detected, such as wherein the label is a partner of a binding pair. By “partner of a binding pair” is meant one of a first and a second moiety, wherein said first and said second moiety have a specific binding affinity for each other. Suitable binding pairs for use in the method include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

Methods for labeling of proteins and compounds with radioisotopes are known in the art. For example, such methods are found in Ohta et al., Molec. Cell 3:535-541 (1999), which is hereby incorporated by reference in its entirety. By “radioisotope” is meant any radioactive molecule. Suitable radioisotopes for use in the method include, but are not limited to ¹⁴C, ³H, ³²P, ³³P, ³⁵S, ¹²⁵I, and ¹³¹I. The use of radioisotopes as labels is well known in the art.

The functionalization of labels with chemically reactive groups such as thiols, amines, carboxyls, etc. is generally known in the art. In some aspects, the label is functionalized to facilitate covalent attachment.

The covalent attachment of the label can be either direct or via a linker. In some aspects, the linker is a relatively short coupling moiety that is used to attach the molecules. A coupling moiety can be synthesized directly onto a component of the method, peptide for example, and contains at least one functional group to facilitate attachment of the label. Alternatively, the coupling moiety can have at least two functional groups, which are used to attach a functionalized component to a functionalized label, for example. In some aspects, the linker is a polymer. In this aspect, covalent attachment is accomplished either directly, or through the use of coupling moieties from the component or label to the polymer. In some aspects, the covalent attachment is direct, that is, no linker is used. In this aspect, the component can contain a functional group such as a carboxylic acid which is used for direct attachment to the functionalized label. It should be understood that the component and label can be attached in a variety of ways, including those listed above. What is important is that manner of attachment does not significantly alter the functionality of the component. For example, in label-peptide, the label should be attached in such a manner as to allow the peptide to be covalently bound to other peptide to form polypeptide chains. As will be appreciated by those in the art, the above description of covalent attachment of a label and peptide applies equally to the attachment of virtually any two molecules of the present disclosure.

### C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits for identifying compounds that interact with HNF4α, the kit comprising an HNF4α antagonist composition and an HNF4α-regulated gene. As another example, the kits can contain HNF4α.

### D. Mixtures

Disclosed are mixtures formed by performing or preparing to perform the disclosed method. For example, disclosed are mixtures comprising an HNF4α antagonist and HNF4α.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### E. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising reagents for detecting HNF4α binding and binding to HNF4α and an electronic instrument for detecting or analyzing HNF4α binding and binding to HNF4α.

### F. Data Structures and Computer Control

Disclosed are data structures used in, generated by, or generated from, the disclosed method. Data structures generally are any form of data, information, and/or objects collected, organized, stored, and/or embodied in a composition or medium. An HNF4α structure stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, use in assays to identify competitive and noncompetitive inhibitors and antagonists of HNF4α, use to treat cancer, use to treat cancer where HNF4α is expressed, use to treat hepatitis B infection, and use to treat conditions involving disregulation of genes regulated by HNF4α. Other uses are disclosed, apparent from the disclosure, and/or will be understood by those in the art.

### Method

Disclosed are methods that use antagonists of HNF4α. For example, disclosed herein is a method of for treating a subject exposed to hepatitis B virus, the method comprising administering to the subject a composition comprising an HNF4α antagonist.

Also disclosed is a method for treating a subject with undesired expression of one or more genes regulated via HNF4α, the method comprising administering to the subject a composition comprising an HNF4α antagonist.

Also disclosed is a method for treating or preventing a metabolic disorder in a subject, the method comprising administering to the subject a composition comprising an HNF4α antagonist.

Also disclosed is a method for identifying compounds that interact with HNF4α, the method comprising bringing into contact a test compound, an HNF4α antagonist, and HNF4α, and detecting unbound HNF4α antagonist, wherein a given amount of unbound HNF4α antagonist indicates a compound that interacts with HNF4α.

Also disclosed is a method for identifying compounds that affect HNF4α regulation, the method comprising bringing into contact BIM5078 and an HNF4α-regulated gene, and detecting changes in the expression of the HNF4α-regulated gene in the presence and absence of a test compound, wherein a difference in expression of the HNF4α-regulated gene in the presence of the test compound relative to expression of the HNF4α-regulated gene in the absence of the test compound indicates a compound that affects HNF4α regulation.

In some forms of the method the subject can exhibit hyperinsulinemia. In some forms of the method the subject can be a neonate. In some forms of the method the subject can have cancer, wherein the cancer expresses HNF4α. In some forms of the method the cancer can be hepatocellular carcinoma. In some forms of the method the cancer can be gastric cancer.

In some forms of the method the composition can be a BIM5078 composition. In some forms of the method the BIM5078 composition can further comprise a detectable agent linked to the BIM5078 compound. In some forms of the method the method can further comprise bringing into contact BIM5078 and an HNF4α-regulated gene, and detecting changes in the expression of the HNF4α-regulated gene in the presence and absence of the compound that interacts with HNF4α, wherein a difference in expression of the HNF4α-regulated gene in the presence of the compound that interacts with HNF4α relative to expression of the HNF4α-regulated gene in the absence of the compound that interacts with HNF4α indicates a compound that affects HNF4α regulation.

In some forms of the method a decrease in the expression of the HNF4α-regulated gene in the presence of the compound that interacts with HNF4α relative to expression of the HNF4α-regulated gene in the absence of the compound that interacts with HNF4α indicates that the compound that interacts with HNF4α inhibits HNF4α. In some forms of the method an increase in the expression of the HNF4α-regulated gene in the presence of the compound that interacts with HNF4α relative to expression of the HNF4α-regulated gene in the absence of the compound that interacts with HNF4α indicates that the compound that interacts with HNF4α decreases inhibition of HNF4α by BIM5078. In some forms of the method the method can further comprise detecting changes in the expression of the HNF4α-regulated gene in the absence of BIM5078 and in the presence and absence of the compound that interacts with HNF4α, wherein an increase in expression of the HNF4α-regulated gene indicates that the compound that interacts with HNF4α increases expression of the HNF4α-regulated gene.

In some forms of the method a decrease in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation inhibits HNF4α. In some forms of the method the HNF4α-regulated gene can express a reporter protein. In some forms of the method an increase in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation decreases inhibition of HNF4α by BIM5078. In some forms of the method the method can further comprise detecting changes in the expression of the HNF4α-regulated gene in the absence of BIM5078 and in the presence and absence of the compound that affects HNF4α regulation, wherein an increase in expression of the HNF4α-regulated gene indicates that the compound that affects HNF4α regulation increases expression of the HNF4α-regulated gene. In some forms of the method the HNF4α-regulated gene can express a reporter protein.

In some forms of the method the metabolic disorder can be a lipid metabolic disorder. In some forms of the method the subject can be hyperlipidemic. In some forms of the method the metabolic disorder can be or can result in hyperlipidemia.

The HNF4α antagonist can be a BIM5078 compound.

The HNF4α antagonist or BIM5078 compound can be a compound having the structure of

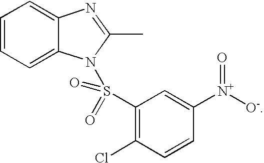

### A. Treating and Administration

Disclosed are methods for treating subject by administrating an HNF4α antagonist. For example, disclosed are methods for treating a subject exposed to hepatitis B virus, for treating a subject with undesired expression of one or more genes regulated via HNF4α, and for treating or preventing a metabolic disorder in a subject. The subject can be treated by administering the HNF4α antagonist to the subject or by administering the HNF4α antagonist to cells ex vivo prior to introduction of the cells to the subject.

In some forms of the method the subject can exhibit hyperinsulinemia. In some forms of the method the subject can be a neonate. In some forms of the method the subject can have cancer, wherein the cancer expresses HNF4α. In some forms of the method the cancer can be hepatocellular carcinoma. In some forms of the method the cancer can be gastric cancer. In some forms of the method the metabolic disorder can be a lipid metabolic disorder. In some forms of the method the subject can be hyperlipidemic. In some forms of the method the metabolic disorder can be or can result in hyperlipidemia.

Examples of metabolic disorders that can be treated with the disclosed HNF4α antagonist include NKT-mediated conditions such as NKT-mediated hepatitis and colitis. As described in Brozovic et al., Nature Medicine 10(5):535-539 (2004), NKT cells require functional CD1d and CD1d function is regulated by microsomal triglyceride transfer protein (MTP). Because the disclosed HNF4α antagonists can reduce MTP function, NKT cell function can be inhibited by the HNF4α antagonists. Thus, disease conditions that require abnormal NKT activity or that require NKY activity can be treated with the disclosed HNF4α antagonist.

A cell can be in vitro. Alternatively, a cell can be in vivo and can be found in a subject. A “cell” can be a cell from any organism including, but not limited to, a bacterium.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. In one aspect, the subject is a mammal such as a primate or a human.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventive treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

In one aspect, the compounds described herein can be administered to a subject comprising a human or an animal including, but not limited to, a mouse, dog, cat, horse, bovine or ovine and the like, that is in need of alleviation or amelioration from a recognized medical condition.

A metabolic disorder is a disorder or condition involving or caused by a change in normal metabolism. Diabetes is an example of a metabolic disorder. In general, too much or too little of one or more metabolic products are produced in a metabolic disorder. For example, too much serum lipids occurs in hyperlipidemia. The underlying genetic, physiologic or biologic cause of hyperlipidemia can vary, but all can be considered part of a metabolic disorder. A metabolic disorder can be caused by genetic mutations or can be caused when some organs, such as the liver or pancreas, become diseased or do not function normally.

By the term “effective amount” of a compound as provided herein is meant a nontoxic but sufficient amount of the compound to provide the desired result. As is discussed further herein, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The term “therapeutically effective” means that the amount of the composition used is of sufficient quantity to ameliorate one or more causes or symptoms of a disease or disorder. Such amelioration only requires a reduction or alteration, not necessarily elimination.

The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject in need HNF4α antagonist for the treatment of HBV, treatment of cancer, treatment of cancer where HNF4α is expressed, and treatment of conditions involving disregulation of genes regulated by HNF4α. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

Further, subjects for administration of the disclosed compounds and compositions can be identified by assessing HNF4α expression and/or activity in the subject and/or in relevant tissues and/or cells of the subject.

By “pharmaceutically acceptable” is meant a material that is not biologically, clinically or otherwise undesirable, i.e., the material can be administered to an individual along with the relevant active compound without causing clinically unacceptable biological effects or interacting in a deleterious manner with any of the other components of the pharmaceutical composition in which it is contained.

Any of the disclosed compounds can be used therapeutically in combination with a pharmaceutically acceptable carrier. The compounds described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The pharmaceutical compositions described herein can include, but are not limited to, carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

The compounds and pharmaceutical compositions described herein can be administered to the subject in a number of ways depending on whether local or systemic treatment is desired, and on the area to be treated. Thus, for example, a compound or pharmaceutical composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

### B. Identification of HNF4α Antagonists

Disclosed are methods for identifying compounds that interact with HNF4α and that affect HNF4α regulation and/or activity. For example, disclosed is a method for identifying compounds that interact with HNF4α, the method comprising bringing into contact a test compound, an HNF4α antagonist, and HNF4α, and detecting unbound HNF4α antagonist, wherein a given amount of unbound HNF4α antagonist indicates a compound that interacts with HNF4α.

Also disclosed is a method for identifying compounds that affect HNF4α regulation, the method comprising bringing into contact BIM5078 and an HNF4α-regulated gene, and detecting changes in the expression of the HNF4α-regulated gene in the presence and absence of a test compound, wherein a difference in expression of the HNF4α-regulated gene in the presence of the test compound relative to expression of the HNF4α-regulated gene in the absence of the test compound indicates a compound that affects HNF4α regulation.

In some forms of the method the composition can be a BIM5078 composition. In some forms of the method the BIM5078 composition can further comprise a detectable agent linked to the BIM5078 compound. In some forms of the method the method can further comprise bringing into contact BIM5078 and an HNF4α-regulated gene, and detecting changes in the expression of the HNF4α-regulated gene in the presence and absence of the compound that interacts with HNF4α, wherein a difference in expression of the HNF4α-regulated gene in the presence of the compound that interacts with HNF4α relative to expression of the HNF4α-regulated gene in the absence of the compound that interacts with HNF4α indicates a compound that affects HNF4α regulation.

In some forms of the method a decrease in the expression of the HNF4α-regulated gene in the presence of the compound that interacts with HNF4α relative to expression of the HNF4α-regulated gene in the absence of the compound that interacts with HNF4α indicates that the compound that interacts with HNF4α inhibits HNF4α. In some forms of the method an increase in the expression of the HNF4α-regulated gene in the presence of the compound that interacts with HNF4α relative to expression of the HNF4α-regulated gene in the absence of the compound that interacts with HNF4α indicates that the compound that interacts with HNF4α decreases inhibition of HNF4α by BIM5078. In some forms of the method the method can further comprise detecting changes in the expression of the HNF4α-regulated gene in the absence of BIM5078 and in the presence and absence of the compound that interacts with HNF4α, wherein an increase in expression of the HNF4α-regulated gene indicates that the compound that interacts with HNF4α increases expression of the HNF4α-regulated gene.

In some forms of the method a decrease in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation inhibits HNF4α. In some forms of the method an increase in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation decreases inhibition of HNF4α by BIM5078. In some forms of the method the method can further comprise detecting changes in the expression of the HNF4α-regulated gene in the absence of BIM5078 and in the presence and absence of the compound that affects HNF4α regulation, wherein an increase in expression of the HNF4α-regulated gene indicates that the compound that affects HNF4α regulation increases expression of the HNF4α-regulated gene. In some forms of the method the HNF4α-regulated gene can express a reporter protein.

An HNF4α antagonist could affect HNF4α activity in a variety of ways. The disclosed uses do not depend on the mechanism of action. However, it can be useful to identify and/or categorize HNF4α antagonists by their effect. The effect of an HNF4α antagonist in respect to HNF4α and the level of DNA binding can be tested by disrupting the binding of HNF4α to its downstream targets. For example, a chromatin immunoprecipitation (ChIP) assay can be done to look for HNF4α bound to the HNF1α promoter, one of HNF4α′ s well documented downstream targets. An example of a ChIP assay is described in Sheena et al., J. Lipid Research 46:328-341 (2005).

### C. Detecting

Binding and dissociation of the disclosed compounds, proteins and other components described herein can be detected using routine methods. In some aspects, one or more components, such as the disclosed compounds and/or HNF4α can include a label. Components having a label can be referred to as “label-X”, wherein X is the component. For example, a peptide comprising a label can be referred to as “label-peptide.” Moreover, reference to a component is also a reference to that component attached to a label. For example, reference to peptide is also a reference to label-peptide, such as His-peptide, which can be used, for example, to isolate, purify, or identify the peptide.

The label can be covalently bound to the attached component. When more than one component of a combination has a label, the labels can be numbered for identification, for example “label1-peptide”. Components can comprise more than one label, in which case each label can be numbered, for example “label1,2-peptide”. Exemplary labels include, but are not limited to, a label, a partner of a binding pair, and a surface substrate binding molecule. As will be evident to the skilled artisan, many molecules can find use as more than one type of label, depending upon how the label is used.

Thus, binding and dissociation of the disclosed compounds, proteins and other components can be detected by detecting labeled compound, protein or component either in the bound state or in the dissociated state. In some aspects, label1 and label2 can be fluorescent labels constituting a fluorescence resonance energy transfer (FRET) pair. In some aspects, detection is performed in a multi-well plate comprising a surface substrate comprising nickel.

Expression of a gene refers to the process of converting genetic information into a functional gene product. Gene expression can include, for example, transcription of a gene, processing of the transcript, translation of the transcript, and processing of the translation product. Changes in gene expression can occur and be measured at any stage of expression. A change in expression, such as an increase or decrease in expression, can be based on a base or reference level or any threshold level of interest. As an example, a gene may be expressed at an abnormally high level and reduction of expression can still be considered a reduction in expression even if the expression only goes back to a normal level (or even if it is not reduced to the normal level).

Numerous methods are known for detecting binding, interaction, and expression levels and such methods can be used for detection purposes set forth herein. Some examples of detection and analytical techniques useful with the disclosed compounds and with HNF4α are described in the examples.

### EXAMPLES

### A. Example 1

### High-Throughput Screen for Probing the Regulation of the Human Insulin Promoter

This example describes a high throughput screen (HTS) for small molecules that can probe the regulation of the human insulin promoter. Also described herein is the result of the HTS, the specific molecule identified from it, and how the HTS and the molecule will apply in systems medicine and the engineering therapeutics using it. The screen identified one molecule, BIM5078, which selectively binds at high affinity to the ligand-binding site in HNF4α and thereby antagonizes HNF4α activity.

1. Materials and Methods

There are over 20 million individuals affected by diabetes in the United States. In 2007, the total cost of diabetes was on the order of 170 billion dollars. Diabetics generally fall into classes: Type I or Type II. Type I diabetes is often characterized by insulin dependency and accounts for anywhere from 5 to 15% of all diabetic cases. It is considered to be an auto-immune disease in which the insulin-secreting β-cells in the pancreas are destroyed. Type II diabetes, however, does not initially require insulin compensation. Instead, it is associated with insulin resistance (often linked to obesity). Even in these patients, it is observed that overt diabetes only occurs when the β-cells fail.

The only cell type in the body that produces insulin is the pancreatic β-cell. The insulin gene, in the β-cell, is controlled by its promoter, located in the 300-400 by region upstream of transcription start site. The insulin promoter is highly regulated by a number of transcription factors. Many of the regulatory networks have been unraveled; however, it has become increasingly clear that many factors remain unknown and unsolved regarding the disease. Because the insulin promoter is a primary target of diabetogenic stimuli, fully understanding its regulation is critical for dissecting the pathways that govern diabetes.

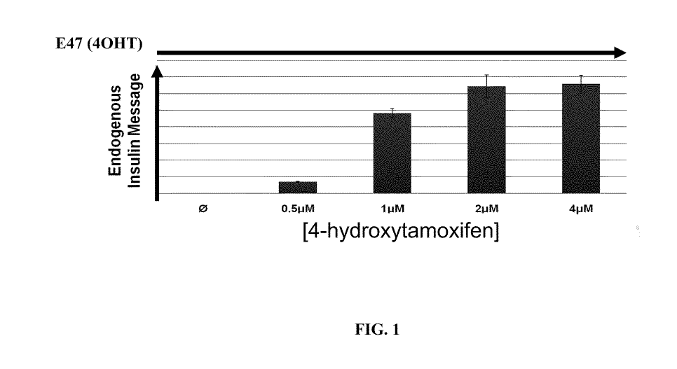

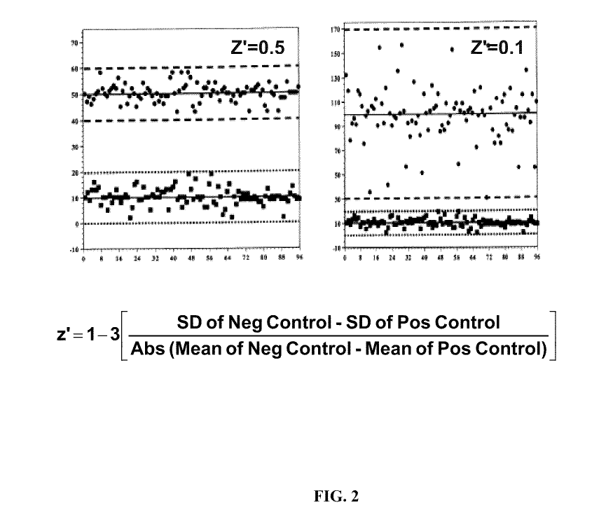

A high-throughput screen (HTS) for compounds that can regulate β-cell differentiated function resulted in the discovery of an HNF4α antagonist. Primary β-cells are in short supply and have a strong tendency to undergo apoptosis when manipulated in culture. Therefore, a β-cell model was used for the studies regarding β-cell growth and differentiation. T6PNE cells were derived from human fetal islets and were engineered to express the transcription factors PDX-1, NeuroD1, and E47, which are all present in the human β-cell. It was observed that induction of E47 upregulates the expression of many genes associated with β-cell function, including insulin. Consistent with what occurs in primary β-cells, it also was observed that E47 induction caused growth arrest in the cell line. Therefore, E47 was engineered to express a modified estrogen receptor in T6PNE cells. In the presence of 4-hydroxytamoxifen (40HT, the fused E47 translocates into the nucleus and becomes transcriptionally active. Insulin gene expression was modulated using T6PNE with inducible E47. Endogenous insulin message in T6PNE cells is upregulated in a dose-dependent manner with E47 induction by 4-hydroxytamoxifen. Titrations were used to measure the mRNA or protein amount of insulin expression. In order to adapt the cell line for HTS assays, a lentiviral vector expressing the insulin promoter-e green fluorescent protein (GFP) cassette ins-GFP transgene was used to infect T6PNE. As a result, compounds that targeted the insulin promoter could be visibly detected by changes in the green channel (observing GFP). FIG. 1 shows a dose-dependent increase in GFP signal resulting from increased E47 induction. This increase correlates with that of the endogenous insulin message. Therefore, expression in the transfunctioned cell can be used as a baseline so both up and down regulators of the insulin promoter can be monitored in the screening assays. Downregulators of the insulin promoter are useful for analyzing the governing pathways in diabetes. However, both up and down modulators were screened for. T6PNE cells were primed by inducing a small amount of E47 prior to compound addition. Insulin expression was monitored and visualized by the ins-GFP transgene. Hits were determined and dependent on the ability to differentiate between a baseline condition and the corresponding positive or negative control. The process is dependent on two main criteria (1) the separation in the dynamic range between baseline and positive or negative control and (2) the variation around the two conditions being compared. These criteria are summarized with a z′ calculation, see the equation in FIG. 2, measuring signal to noise ratio. A lower z′ values suggests more replicates are required in the screening process.

The HTS was performed in a 384-well format where primed T6PNE cells express the ins-GFP transgene and the test compound was added 24 hours later. After 48 hours of incubation the plates were fixed and imaged using high throughput microscopy. The Images were then analyzed using algorithms (Jeff Price at the Screening Center at the Burnham Institute). Images of each well were captured in the blue channel (to evaluate cell number), the green channel (a readout for insulin gene expression) and the red channel (to eliminate autofluorescent compounds). DAPI, a fluorescent stain, that binds tightly to DNA produces a punctuate nuclear mask that allows the microscope to focus from well to well on the plate. It is also used to segment the images to perform cell by cell analysis. Furthermore, hits are determined by applying threshold intensity gates on the green channel and counting the number of cells above or below that gate in a given well. These counts are then normalized to the total number of cells per well. The compounds that were indicated as hits were subjected to a number of confirmatory assays, including dose responsiveness and affect on endogenous insulin message. Upon confirmation the compounds underwent mechanistic studies to determine pathways modulated. Compounds from a diverse small molecule library were screened and the molecule known as “BIM5078” was shown to be a downregulator of human insulin promoter activity. BIM5078 was shown to have properties that could be used to treat, for example, Hepatitis B, metabolic disorders, and cancer—such as gastric and hepatocellular carcinoma.

The core structure of BIM5078, benzimidazole, is related to known PPARγ compounds that functions as agonists or antagonists. BIM5078 was used in a PPAR response element (PPRE)-luciferase reporter assay. HeLa cells were co-transfected with a PPAR response element (PPRE) reporter plasmid and a human PPARγ expression vector. BIM5078 activated the PPRE in HeLa cells and activation was enhanced by co-transfection with a PPARγ expression vector, thereby, confirming the effect of BIM5078 as a PPARγ agonist. Known PPARγ agonists were used as positive controls. The same PPARγ agonist compounds were tested on T6PNE ins-eGFP for their ability to repress the insulin promoter. Known PPARγ agonists are unable to suppress insulin gene transcription. Similarly, other PPAR agonists of the alpha and delta class were also negative in the assay. However, because HNF4α also binds to PPREs, BIM5078's ability to bind HNF4α was further investigated.

2. Results and Discussion

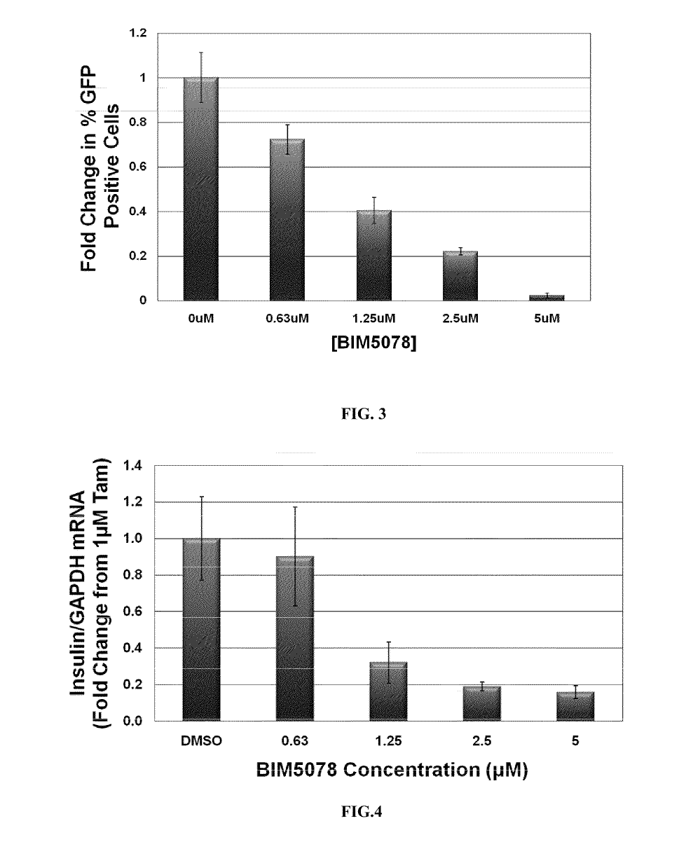

The HTS resulted in identification of a number of activators and repressors. A few of these were selected for further evaluation. Several compounds were first indicated as hits; however, upon confirmation only one compound, BIM5078, was confirmed as a true hit. Two confirmation assays were performed. The initial confirmatory assay of BIM5078 was performed by measuring the readout of the insulin-GFP transgene. BIM5078 at 5 μM was found to potently suppress GFP expression up to 30-fold; suppression by BIM5078 was a dose-dependent (FIG. 3). The second assay confirmed the dose-responsiveness on endogenous insulin message. In this assay 5 μM BIM5078 mediated a 6-fold repression of insulin message induced by treatment with 1 μM 4-hydroxytamoxifen (FIG. 4).

### B. Example 2

### Binding of BIM5087 to HNF4α Receptor

This example describes examination of binding of BIM5078 to HNF4α.

1. Materials and Methods

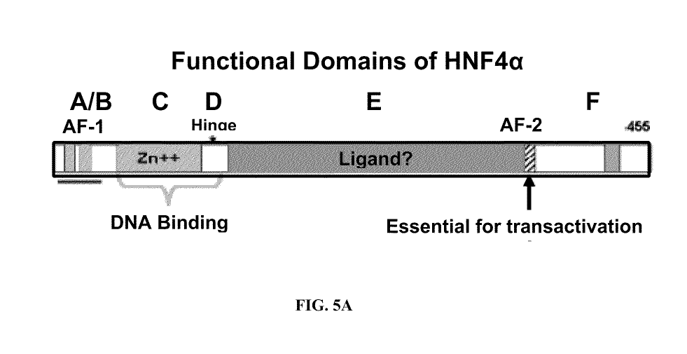

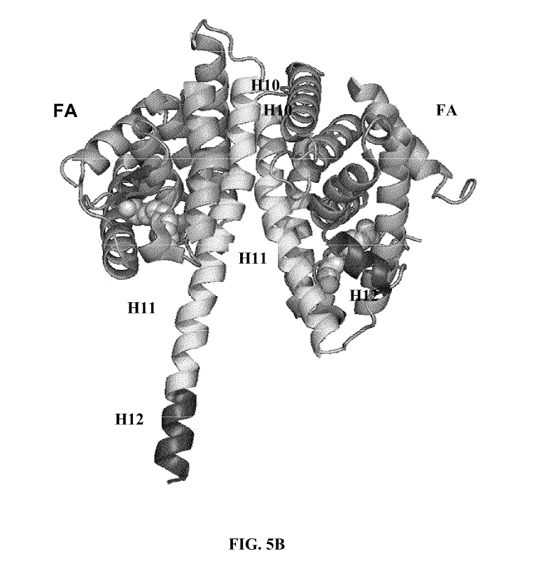

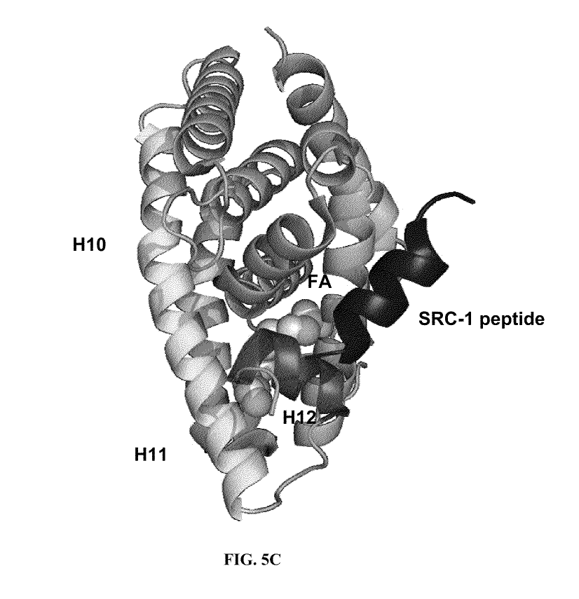

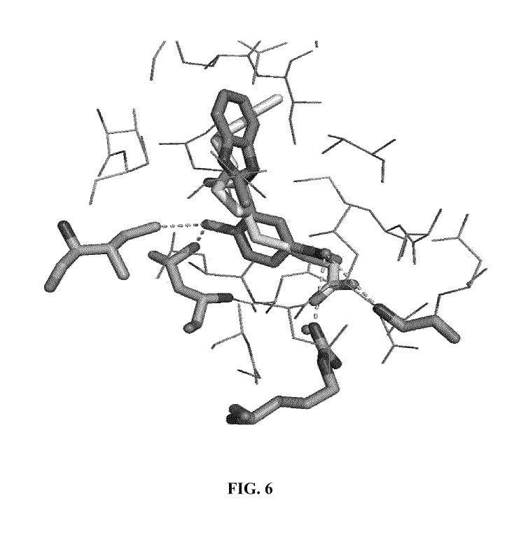

HNF4α is a member of the nuclear receptor family found only in the pancreas, liver, kidney, colon, small intestine and testes (Drewes et al. Molecular and Cellular Biology, 1996, 925-931). HNF4α has been implicated in a number of disease states, including diabetes, cancer, hepatitis, hemophilia, thrombosis, hypoxia anemia, and atherosclerosis. For example, mutations in HNF4α result in a form of autosomal dominant diabetes known as MODY1 (maturity onset-diabetes of the young). The functional domains of HNF4α resemble traditional members of the nuclear receptor superfamily, yet it only binds as a homodimer and only to DNA response elements consisting of direct repeats. HNF4α has been shown to activate transcription in the absence of exogenous ligand. Crystallization of its LBD yielded a protein with fatty acids in the ligand-binding pocket, even in the case when no free fatty acid (FFA) was added. The crystal structure of rat HNF4α complexed with FFA can be seen in FIG. 5 (Duda et al. Structural Basis for HNF-4α Activation by Ligand and Coactivator Binding, J. Biol. Chem. 279:23311-23316). Although the FFA is present in each monomer of the homodimer, half of the complex remains in the open (inactive) conformation. In the open conformation helix 12 is located away from the ligand-binding pocket; while in the closed conformation it caps the pocket. This indicates that there is a regulatory component in addition to the ligand that is involved in the activation of HNF4α. In a similar study using human HNF4α, it was found that a peptide containing the NR box motif of SRC-1 (a co-activator of HNF4α) enables helix 12 to maintain the active state. The direct binding assay indicates that BIM5078 binds HNF4α protein with a very low dissociation constant of 12 nM. This strongly indicates that BIM5078 is acting on HNF4α as its true pharmacologic target.

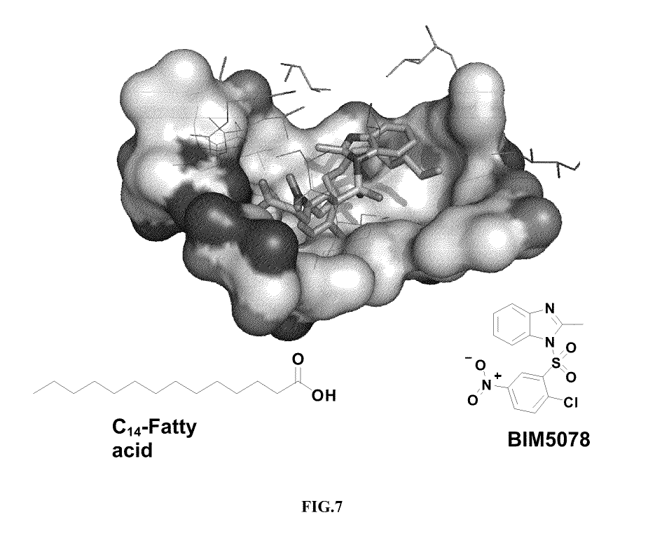

To evaluate the functional activity of BIM5078, the effect of HNF4α siRNA on the human insulin promoter was evaluated. The siRNA to HNF4α decreased the % of GFP positive cells (an indicator of insulin promoter activity) nearly 15-fold. This indicated that BIM5078 was behaving as an HNF4α antagonist. In silico docking of BIM5078 into the HNF4α LBD as found in the crystal structure of its complexes with fatty acids (PDB Code: 1m7w and pzl) using BioMedCache occurred in the binding pocket occupied by the presumptive ligand (free fatty acids). The superposition of the docking pose of BIM5078 with the original ligand (fatty acid) clearly supports the observation that BIM5078 displaces the fatty acid from the ligand-binding pocket (FIG. 6). Especially intriguing is the chloro group of BIM5078, which docking suggests would have a hydrophobic interaction with residue Valine 178 (FIG. 7). The high affinity binding of BIM5078 to HNF4α leads to repression of insulin promoter activity. A similar effect on the promoter occurs in the presence of siRNA to HNF4α.

Many ligands for nuclear receptor subfamilies induce the translocation of their receptors between the cytoplasm and the nucleus. HNF4α is believed to be localized entirely in the nucleus and this is the case even upon the addition of BIM5078.

In the literature a controversy exists over the HNF4α binding site in the human insulin promoter. Considerable variation occurs in the consensus sequence for HNF4α(see notes at the end of Table 1). The element on the insulin promoter may be one such variation.

Numerous distinct target genes have been identified as targets for HNF4α. Many of the target genes contain more than one HNF4α binding site. HNF4α binding sites generally are direct repeats of 5-AGGTCA-3″ with a spacing of one nucleotide between the repeats (DR-1). HNF4α is also known to bind sites with 2 nucleotides between the repeats (DR2 sites). HNF4α does not bind sites with other spacings. Some significant variation from the consensus repeat is seen in one of the repeats in a pair. HNF4α binding sites can often be identified by three adenines in the middle of the site. HNF4α binding sites are highly conserved between species. The consensus for the HNF4 binding site is:

Direct Repeat 1 (DR1g)

(SEQ ID NO: 1)

5′-AGGTCA g AGGTCA-3′
Direct Repeat 2 (DR2aa)

(SEQ ID NO: 2)

| | |
|---|---|
| 5′-AGGTCA aa AGGTCA-3′ | |
| Consensus | G G G T C A A A G G T C A |
| (SEQ ID NO: 3) | A t C t g g T C t g |
| | a G g c |
| | t |

Upper case letters indicate nucleotides that appear in the indicated position in 25% or more of sites. Lower case letters indicate nucleotides that appear in the indicated position in 10 to 25% of the sites.

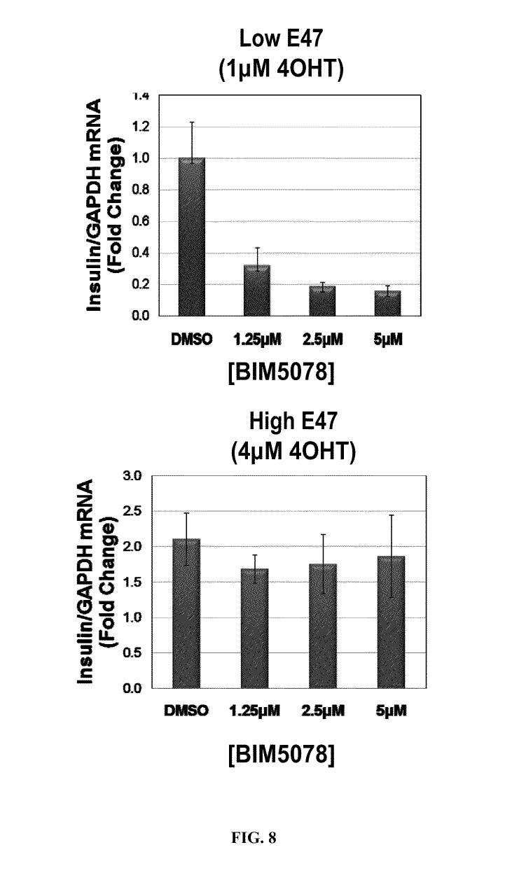

HNF1α is a downstream target of HNF4α and has a known binding site on the human insulin gene promoter. BIM5078 was also shown to decrease the HNF1α transcription. It is unclear whether HNF4α is modulating the insulin promoter directly or indirectly through a downstream target. Overexpression of HNF1α or HNF4α would overcome the repressive effects of BIM5078. The repressive effect of BIM5078 was lost when it was added to T6PNE cells with high levels of induced E47 (FIG. 8). High E47 levels are thought to resemble the normal state of a β-cell while low levels of E47 to simulate the stress condition.

Fatty acids are the presumptive ligands for HNF4α and support a role for fat metabolism and lipotoxicity, with the latter being an important pathway in the pathogenesis of type 2 diabetes. Glucose and fatty acids are believed to act through a number of intermediates, including reactive oxygen species (ROS) and ceramides, to suppress insulin promoter activity. They ultimately disrupt the transcriptional machinery that drives insulin gene expression, in a process known as lipotoxicity. For example, the addition of a saturated fatty acid, such as palmitic acid or its palmitate salt, suppresses the insulin promoter activity in a dose-dependent fashion. The monounsaturated fatty acid, oleic acid or its oleate salt, has a similar repressive effect. BIM5078 was shown to be an HNF4α antagonist that decreases the insulin promoter activity. A similar effect is observed when the presumptive ligand for HNF4α is added to T6PNE cells. These effects, however, are lost at high levels of insulin gene expression. A model based on these facts indicates that HNF4α protects the n-cell from lipotoxic stress by stabilizing transcriptional machinery on the insulin promoter.

### C. Example 3

### BIM5078 Down-Regulation of Hepatitis B Linked to the HNF4α Receptor

This example describes the effect of HNF4α antagonists on HBV and the importance of HNF4α to Hepatitis B virus replication. BIM5078 was shown to reduce the amount of surface antigen, believed to reflect viral production, as measured using the Wild-Type HBV construct (pWTD).

1. Methods and Materials

Hepatitis is an inflammation of the liver, resulting in liver cell damage and destruction. Hepatitis B virus can lead to cirrhosis, liver cancer, liver failure, and death. Despite the existence of a vaccine, hepatitis B is still a significant problem across the globe and there are an estimated 300 million carriers worldwide, 1.5 million of which are in the US.

The hepatitis B virus belongs to a family of DNA viruses called Hepadnaviridae which primarily infect liver cells. There are four known genes encoded by the genome called C, S, P and X. Two categories of drugs are used in HBV therapy: (1) interferon, a synthetic version of antiviral proteins produced by the immune system, and (2) specific inhibitors of the reverse-transcriptase function of HBV-DNA polymerase. Similar to what occurs with HIV, mutations in HBV result in antiviral resistance. Therefore, applying combination therapy can be just as useful for HBV as it has been for HIV.

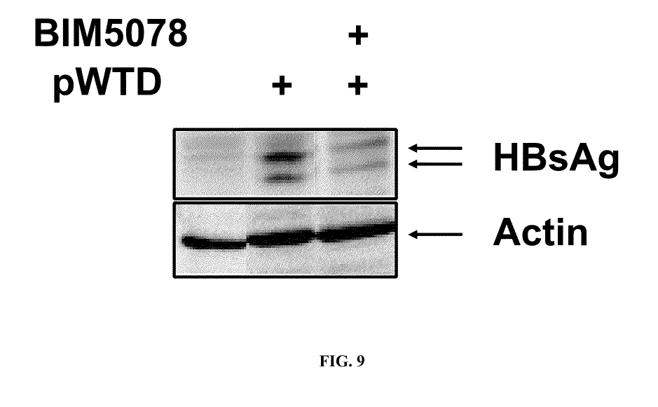

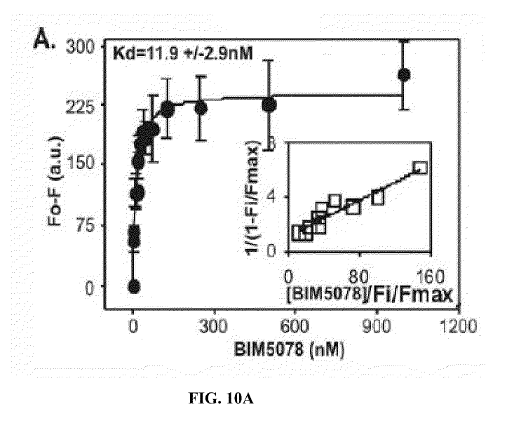

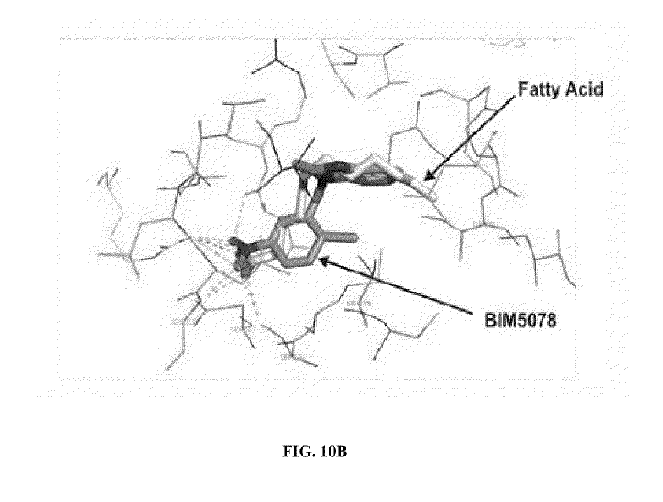

BIM5078 is an HNF4α antagonist and can be useful in treatment of diseases involving HNF4α modulation. There are a number of HNF4α binding sites in the HBV genome, including the enhancer region, the core promoter and the polymerase. BIM5078 can downregulate the core and polymerase, and it has been discovered that it can effectively suppress or eliminate HBV proliferation. Also, the HNF4α binding sites are well-conserved and no mutations of these sites have yet been discovered in the various HBV strains. Thus, targeting HNF4α can circumvent the resistance seen with traditional therapies. It has previously been demonstrated that there is fundamental link between HNF4α and HBV replication (Quasdorff et al. Cellular Microbiology, 2008, 10(7), 1478-1490). Knocking-down HNF4α using siRNA effectively reduced HBV core protein levels in a hepatoma cell line stably expressing HBV. This indicates that HNF4α inhibition can prevent virus production. The ability of the HNF4α antagonist BIM5078 to reduce viral production in Huh7 cells transfected with the wild-type HBV construct (pWTD) was tested. Addition of BIM5078 to the transfected cells decreased their levels of surface antigen, as detected by staining. The large decrease in the two glycosylation forms of the surface antigen was visualized as doublet bands (FIG. 9). Actin levels, however, showed no dependence on BIM5078.

### D. Example 4

### Compound Synthesis

Disclosed compounds can be synthesized using any suitable methods. Examples of useful synthetic schemes are illustrated below.

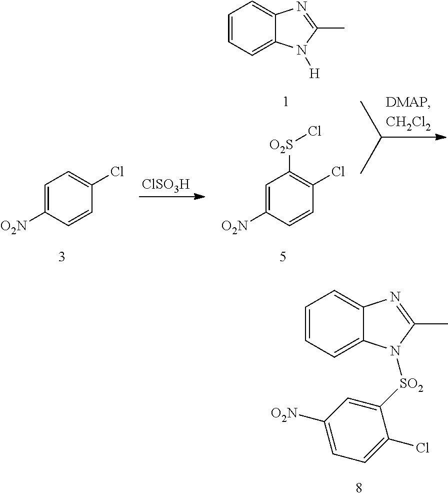

General Procedure for 1-Phenylsulfonyl-1H-benzo[d]imidazoles. To a suspension of the benzimidazole (1.0 mmol) and aryl-1-sulfonyl chloride (1.0 mmol) in dry CH₂Cl₂ (10 mL) was added 4-(dimethylamino)pyridine (122 mg, 1.0 mmol). The clear solution was stirred at 50° C. for 10 h, cooled to room temperature, and partitioned between CH₂Cl₂ and water. The organic layer was washed, dried, and concentrated at reduced pressure. The crude product was crystallized (ethyl acetate) to give the 1-arylsulfonyl-1H-benzo[d]imidazole.

2-Chloro-5-nitrobenzene-1-sulfonyl Chloride (5). [1, 2] 1-Chloro-4-nitrobenzene (3) (158 mg, 1.0 mmol) in chlorosulfonic acid (10 mL) was stirred at 120° C. for 10 h, cooled to room temperature, and poured slowly with stirring into ice-water. The resulting precipitate was filtered, dried under vacuum and then crystallized (hexane).to afford 5 as red needles (195 mg, 76%), mp 78-80° C.; FTIR 1599, 1529, 1347, 1182 cm⁻¹; ¹H NMR (CDCl₃) δ 7.89 (d, J=9.0, 1H), 8.52 (dd, J=9.0, 2.1 Hz, 1H), 9.00 ppm (d, J=2.1 Hz, 1H). CAS 4533-95-3 commercially available, cited.

1-(2-Chloro-5-nitrophenylsulfonyl)-2-methyl-1H-benzo[d]imidazole (8, BIM-5078, BI-6005). A suspension of 2-methylbenzimidazole (1) (132 mg, 1.0 mmol) and 2-chloro-5-nitrobenzenesulfonyl chloride (5) gave after chromatography (33% EtOAc/hexane) 8 (296 mg, 84%) as a tan solid, mp 196-198° C.; FTIR 1601, 1532, 1348, 1245, 1178 cm⁻¹; ¹H NMR (DMSO-d₆) δ 2.75 (s, 3H), 7.30 and 7.36 (2dd, J=8.7, 7.2 Hz, 2H), 7.57 and 7.68 (2d, J=7.5 Hz, 2H), 8.01 (d, J=8.7 Hz, 1H), 8.58 (dd, J=8.7, 2.7 Hz, 1H), 8.91 ppm (d, J=2.7 Hz, 1H). HRMS calcd C₁₄H₁₀ClN₃O₄S [M+H]⁺ 352.0153, found 352.0151. CAS 337506-43-1, not cited, commercially available.

### 1. CITATIONS

[1] Smith, G. C. M.; Martin, N. M. B.; Cockcroft, X. F.; Matthews, I. T. M.; Menear, K. A.; Rigoreau, L. J. M.; Hummersone, M. G.; Griffin, R. J. (Kudos Pharmaceuticals Limited, UK). Preparation of aminopyrones for use in pharmaceutical compositions as ATM kinase inhibitors. WO Patent 2005/016919 (Feb. 24, 2005).
[2] Raiford, L.; Grosz, O. Alkyl- and aryl-sulfonyl derivatives of o-aminophenols. J. Am. Chem. Soc. 53: 3420-3426, 1931.
[3] Fotsch, C. H.; Tasker, A. Phenylethanine derivatives as calcium receptor modulating agents and their preparation, pharmaceutical compositions and use in the treatment of diseases. World Patent WO 2008057282 A1 20080515.
[4] Courtin, A. Zur sulfonieter Derivate von 4-Fluoranilin. Hely. Chim. Acta 65, 546-550, 1982.
[5] Fel'dman, Kh.; Mikheeva, L. F. Amino sulfides and amino sulfones. XXXI. Synthesis of some sulfonamides with heterocyclic radicals at the sulfonyl group. Zh. Obschchei Khim. 33: 2976-2980, 1963.
Ragno R., et al. J. Med. Chem. 49: 3172-3184, 2006.
Ragno, R., et al. J. Med. Chem. 48: 213-223, 2005.
Artico, M., et al. J. Med. Chem. 39: 522-530, 1996.
Artico, M., et al. Preparation of 1H-pyrrol-1-yl- and 1H-indol-1-yl aryl sulfones for treatment of HIV-1 infections. World Patent WO9633171 A1 19961024.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a compound” includes a plurality of such compounds, reference to “the compound” is a reference to one or more compounds and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

We claim:

1. A method for treating a subject exposed to hepatitis B virus, the method comprising
administering to the subject a composition comprising a compound having the structure 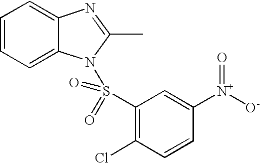
or a pharmaceutically acceptable salt or acid form thereof.

2. A method for treating a subject with undesired expression of one or more genes regulated via HNF4α, the method comprising
administering to the subject a composition comprising a compound having the structure 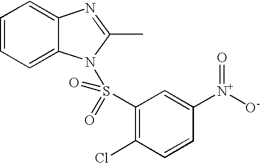
or a pharmaceutically acceptable salt or acid form thereof.

3. The method of claim 2, wherein the subject exhibits hyperinsulinemia.

4. The method of claim 2, wherein the subject is a neonate.

5. The method of claim 2, wherein the subject has cancer, wherein the cancer expresses HNF4α.

6. The method of claim 5, wherein the cancer is hepatocellular carcinoma.

7. The method of claim 5, wherein the cancer is gastric cancer.

8. A method for identifying compounds that interact with HNF4α, the method comprising
bringing into contact a test compound, a BIM5078 composition, and HNF4α, and
detecting unbound BIM5078 composition, wherein a given amount of unbound BIM5078 composition indicates a compound that interacts with HNF4α.

9. The method of claim 8, wherein the BIM5078 composition further comprises a detectable agent linked to the BIM5078.

10. The method of claim 8 further comprising bringing into contact BIM5078 and an HNF4α-regulated gene, and
detecting changes in the expression of the HNF4α-regulated gene in the presence and absence of the compound that interacts with HNF4α, wherein a difference in expression of the HNF4α-regulated gene in the presence of the compound that interacts with HNF4α relative to expression of the HNF4α-regulated gene in the absence of the compound that interacts with HNF4α indicates a compound that affects HNF4α regulation.

11. The method of claim 10, wherein a decrease in the expression of the HNF4α-regulated gene in the presence of the compound that interacts with HNF4α relative to expression of the HNF4α-regulated gene in the absence of the compound that interacts with HNF4α indicates that the compound that interacts with HNF4α inhibits HNF4α.

12. The method of claim 10, wherein an increase in the expression of the HNF4α-regulated gene in the presence of the compound that interacts with HNF4α relative to expression of the HNF4α-regulated gene in the absence of the compound that interacts with HNF4α indicates that the compound that interacts with HNF4α decreases inhibition of HNF4α by BIM5078.

13. The method of claim 10 further comprising detecting changes in the expression of the HNF4α-regulated gene in the absence of BIM5078 and in the presence and absence of the compound that interacts with HNF4α, wherein an increase in expression of the HNF4α-regulated gene indicates that the compound that interacts with HNF4α increases expression of the HNF4α-regulated gene.

14. A method for identifying compounds that affect HNF4α regulation, the method comprising
bringing into contact BIM5078 and an HNF4α-regulated gene, and
detecting changes in the expression of the HNF4α-regulated gene in the presence and absence of a test compound, wherein a difference in expression of the HNF4α-regulated gene in the presence of the test compound relative to expression of the HNF4α-regulated gene in the absence of the test compound indicates a compound that affects HNF4α regulation.

15. The method of claim 14, wherein a decrease in the expression of the HNF4α-regulated gene in the presence of the compound that affects HNF4α regulation relative to expression of the HNF4α-regulated gene in the absence of the compound that affects HNF4α regulation indicates that the compound that affects HNF4α regulation inhibits HNF4α.

16. The method of claim 14, wherein an increase in the expression of the HNF4α-regulated gene in the presence of the compound that affects HNF4α regulation relative to expression of the HNF4α-regulated gene in the absence of the compound that affects HNF4αregulation indicates that the compound that affects HNF4α regulation decreases inhibition of HNF4α by BIM5078.

17. The method of claim 16 further comprising detecting changes in the expression of the HNF4α-regulated gene in the absence of BIM5078 and in the presence and absence of the compound that affects HNF4α regulation, wherein an increase in expression of the HNF4α-regulated gene indicates that the compound that affects HNF4α regulation increases expression of the HNF4α-regulated gene.

18. The method of claims 10, wherein the HNF4α-regulated gene expresses a reporter protein.

19. A method for treating or preventing a metabolic disorder in a subject, the method comprising
administering to the subject a composition comprising a compound having the structure 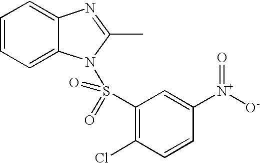
or a pharmaceutically acceptable salt or acid form thereof.

20. The method of claim 19, wherein the metabolic disorder is a lipid metabolic disorder.

21. The method of claim 19, wherein the subject is hyperlipidemic.

22. The method of claims 19, wherein the metabolic disorder is or results in hyperlipidemia.


SEQUENCE LISTING (3 sequences)

SEQ ID NO: 1 - DNA, 13 bp, Homo sapiens
  promoter (1)..(13)
aggtcagaggtca

SEQ ID NO: 2 - DNA, 14 bp, Homo sapiens
  promoter (1)..(14)
aggtcaaaaggtca

SEQ ID NO: 3 - DNA, 13 bp, Homo sapiens
  promoter (1)..(13): Consensus sequence of HNF4alpha
rgdbyarrgkbyn